(19) United States
(12) Patent Application Publication
(10) Pub. No.: US 2026/0130666 A1
(43) Pub. Date: May 14, 2026

(54) END EFFECTOR WITH ENHANCED GRIP AND RELATED METHODS

(71) Applicant: Cilag GmbH International, Zug (CH)

(72) Inventors: Austin E. Wise, Cincinnati, OH (US); Konstantin G. Zabotkin, Blue Ash, OH (US)

(21) Appl. No.: 19/313,323

(22) Filed: Aug. 28, 2025

Related U.S. Application Data
(60) Provisional application No. 63/718,027, filed on Nov. 8, 2024.

(51) Int. Cl. A61B 17/062; A61B 34/30

(52) U.S. Cl. CPC A61B 17/0625; A61B 2034/305; A61B 2560/04

(57) ABSTRACT

A surgical instrument includes a first and second jaws. The first jaw has a first interior surface and first side surface such that the first side surface defines a first groove configured to accept a suture thread within and inhibit the suture thread from traversing along the first side surface in a longitudinal direction. The second jaw has a second interior surface and a second side surface such that the second side surface defines a second groove configured to accept the suture thread within and inhibit the suture thread from traversing along the second side surface in the longitudinal direction. The first and second jaws are movable relative to each other from an open state toward a closed state. In the closed state, the first interior surface and the second interior surface are configured to cooperate with each other to securely grip a needle or the suture thread.

### CROSS-REFERENCE TO RELATED APPLICATIONS

This non-provisional application claims the benefit of priority to U.S. Provisional Patent Application No. 63/718,027, filed on Nov. 8, 2024, the entirety of which is incorporated herein by reference.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probe, etc. Each of these structures performs functions for the surgeon, for example, cutting tissue, coagulating tissue, holding or driving a needle, grasping a blood vessel, dissecting tissue, cauterizing tissue, and/or other functions.

As noted above, the surgeon may employ a needle driver to manipulate a needle. In some instances, the surgeon may desire to employ the same needle driver to manipulate a suture. In such cases, the suture thread may at least partially wrap around the side surfaces of the needle driver in order to tie a knot or the like with the suture thread. When the suture thread is manipulated in this way, it may undesirably move in a proximal or distal direction along the lateral surfaces of the needle driver. The needle drivers of the present disclosure seek to provide structures and methods to reduce or prevent undesired proximal or distal movement of the suture thread at least partially wrapped around the lateral surfaces of the needle driver by including grooves on the lateral surfaces of the needle driver and/or treating the lateral surfaces of the needle driver with coatings, textures, or surface treatments to increase the coefficient of friction along these surfaces.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first illustrative robotic system configured for a laparoscopic procedure;

FIG. 2 depicts a perspective view of a second illustrative robotic system;

FIG. 3 depicts an end elevational view of the robotic system of FIG. 2;

FIG. 4 depicts an end elevational view of the robotic system of FIG. 2 including an illustrative pair of robotic arms;

FIG. 5 depicts a partially exploded perspective view of the robotic arm of FIG. 4 having an instrument driver and a first illustrative surgical instrument;

FIG. 6A depicts a side elevational view of the surgical instrument of FIG. 5 in a retracted position;

FIG. 6B depicts a side elevational view the surgical instrument of FIG. 5 in an extended position;

FIG. 7A depicts an enlarged perspective view of a second illustrative surgical instrument, more particularly a grasper instrument;

FIG. 7B depicts another enlarged perspective view of the grasper instrument of FIG. 7A, with a distal clevis of the grasper instrument illustrated as transparent to show certain internal features thereof;

FIG. 7C depicts a side elevational view of the grasper instrument of FIG. 7A;

FIG. 7D depicts another side elevational view of the grasper instrument of FIG. 7A;

FIG. 7E depicts a top plan view of a proximal clevis of the grasper instrument of FIG. 7A;

FIG. 8 depicts an enlarged perspective view of a third illustrative surgical instrument, more particularly, a needle driver instrument;

FIG. 9 depicts an enlarged perspective view of an example of an end effector of the needle driver instrument of FIG. 8;

FIG. 10 depicts an enlarged perspective view of a distal end of a jaw pair of the end effector of FIG. 9;

FIG. 11 depicts an exploded view with one of the jaws of the jaw pair rotated relative to the other jaw of the jaw pair of FIG. 10;

FIG. 12 depicts a side view of the jaw pair of FIG. 10 in an open position;

FIG. 13 depicts a side view of the jaw pair of FIG. 10 in a closed position;

FIG. 14 depicts a zoomed perspective view of the jaw pair shown in FIG. 10 directing a suture needle and suture thread;

FIG. 15 depicts a portion of a top view of an example of a jaw to be used in a grasper instrument;

FIG. 16 depicts a portion of a top view of an example of a jaw to be used in a grasper instrument;

FIG. 17 depicts a portion of a top view of an example of a jaw to be used in a grasper instrument;

FIG. 18 depicts a portion of a top view of an example of a jaw to be used in a grasper instrument;

FIG. 19 depicts a portion of a top view of an example of a jaw to be used in a grasper instrument;

FIG. 20 depicts a portion of a top view of an example of a jaw to be used in a grasper instrument; and

FIG. 21 depicts an enlarged perspective view of a distal end of an example of a jaw pair of an end effector.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Example of Robotically-Enabled Medical System

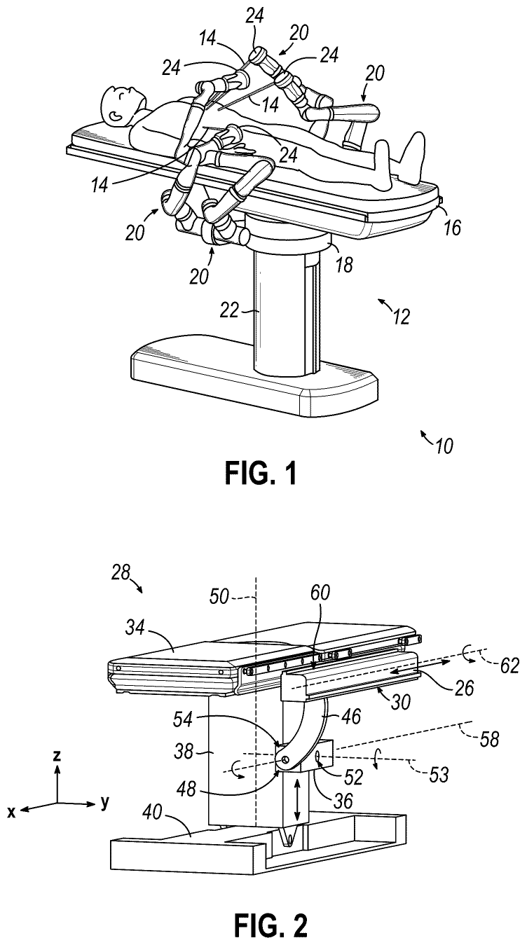

FIG. 1 shows an example of a robotically-enabled medical system, including a first example of a robotic system (10). Robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of robotic system (10) may be constructed and operable in accordance with at least some of the teachings of any of the various patents, patent application publications, and patent applications that are cited herein.

### A. Example of Robotic System With Annular Carriage

As shown in FIG. 1, robotic system (10) includes table system (12) having a platform, such as a table (16), with a plurality of carriages (18) which may also be referred to herein as “arm supports,” respectively supporting the deployment of a plurality of robotic arms (20). Robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

Robotic system (10) may also include a tower (not shown) that divides the functionality of robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation.

### B. Example of Robotic System With Bar Carriage

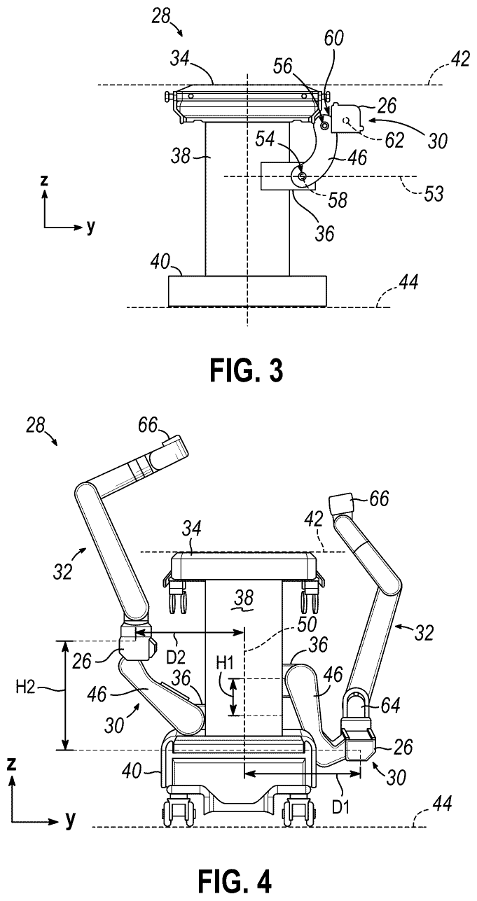

FIGS. 2-4 show another example of a robotic system (28). Robotic system (28) of this example includes one or more adjustable arm supports (30) including bars (26) that are configured to support one or more robotic arms (32) (see FIG. 4) relative to a table (34). In the present example, a single adjustable arm support (30) (FIGS. 2-3) and a pair of adjustable arm supports (30) (FIG. 4) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 4 shows a version of robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 4, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

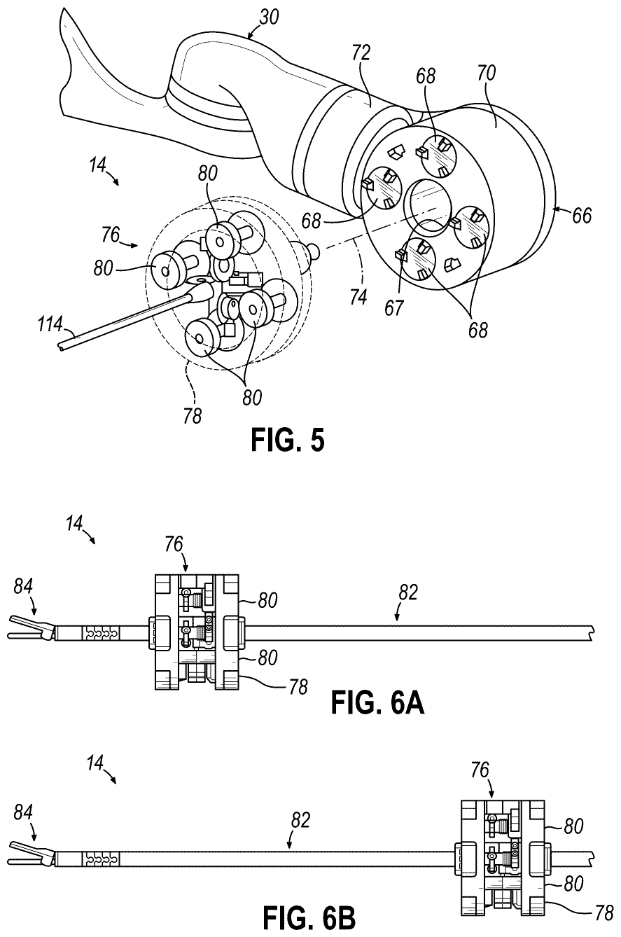

FIG. 5 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

### C. Example of Surgical Instrument With Instrument-Based Insertion Architecture

FIGS. 5-6B show surgical instrument (14) having the instrument-based insertion architecture as discussed above. Surgical instrument (14) includes an elongated shaft assembly (82), an end effector (84) connected to and extending distally from shaft assembly (82), and an instrument base (76) (shown with a transparent external skin for discussion purposes) coupled to shaft assembly (82). Instrument base (76) includes an attachment surface (78) and a plurality of drive inputs (80) (such as receptacles, pulleys, and spools) configured to receive and couple with respective rotary drive outputs (68) of instrument driver (66). Insertion of shaft assembly (82) is grounded at instrument base (76) such that end effector (84) is configured to selectively move longitudinally from a retracted position (FIG. 6A) to an extended position (FIG. 6B), vice versa, and any desired longitudinal position therebetween. As used herein, the retracted position is shown in FIG. 6A and places end effector (84) relatively close and proximally toward instrument base (76); whereas the extended position is shown in FIG. 6B and places end effector (84) relatively far and distally away from instrument base (76). Insertion into and withdrawal of end effector (84) relative to the patient may thus be facilitated by surgical instrument (14), although it will be appreciated that such insertion into and withdrawal may also occur via adjustable arm supports (30) in one or more examples.

When coupled to rotational assembly (70) of instrument driver (66), surgical instrument (14), comprising instrument base (76) and instrument shaft assembly (82), rotates in combination with rotational assembly (70) about the instrument driver axis (74). Since instrument shaft assembly (82) is positioned at the center of instrument base (76), instrument shaft assembly (82) is coaxial with instrument driver axis (74) when attached. Thus, rotation of the rotational assembly (70) causes instrument shaft assembly (82) to rotate about its own longitudinal axis. Moreover, as instrument base (76) rotates with instrument shaft assembly (82), any tendons connected to drive inputs (80) of instrument base (76) are not tangled during rotation. Accordingly, the parallelism of the axes of rotary drive outputs (68), rotary drive inputs (80), and instrument shaft assembly (82) allows for the shaft rotation without tangling any control tendons, and clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

In addition to the foregoing, robotic systems (10, 28) may be configured and operable in accordance with at least some of the teachings of U.S. Pat. No. 9,737,371, entitled “Configurable Robotic Surgical System with Virtual Rail and Flexible Endoscope,” issued Aug. 22, 2017, the disclosure of which is incorporated by reference herein, in its entirety; U.S. Pat. No. 10,945,904, entitled “Tilt Mechanisms for Medical Systems and Applications,” issued Mar. 16, 2021, the disclosure of which is incorporated by reference herein, in its entirety; U.S. Pub. No. 2019/0350662, entitled “Controllers for Robotically-Enabled Teleoperated Systems,” published Nov. 21, 2019, the disclosure of which is incorporated by reference herein, in its entirety; U.S. Pub. No. 2020/0085516, entitled “Systems and Methods for Concomitant Medical Procedures,” published Mar. 19, 2020; and/or U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety.

### II. Surgical Instrument With Grasper

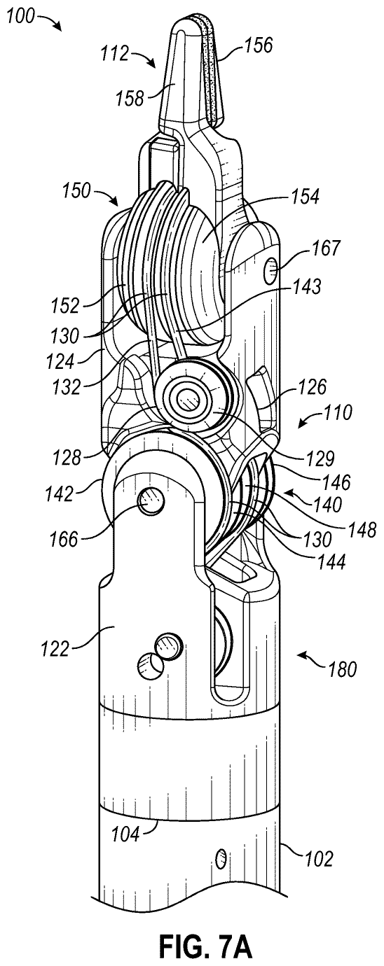

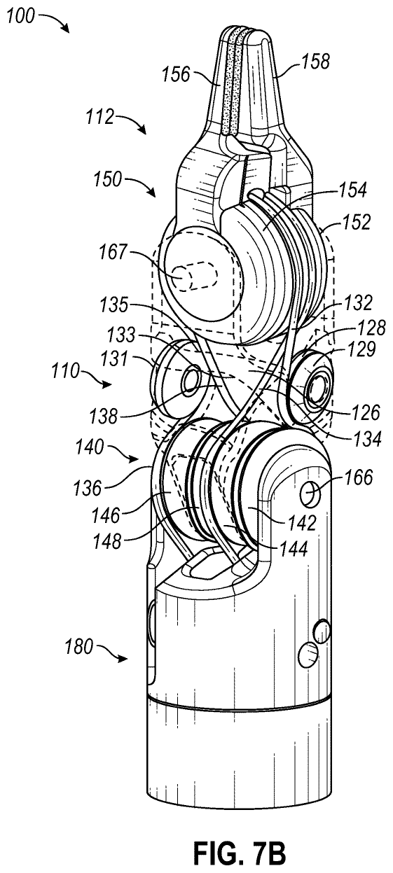

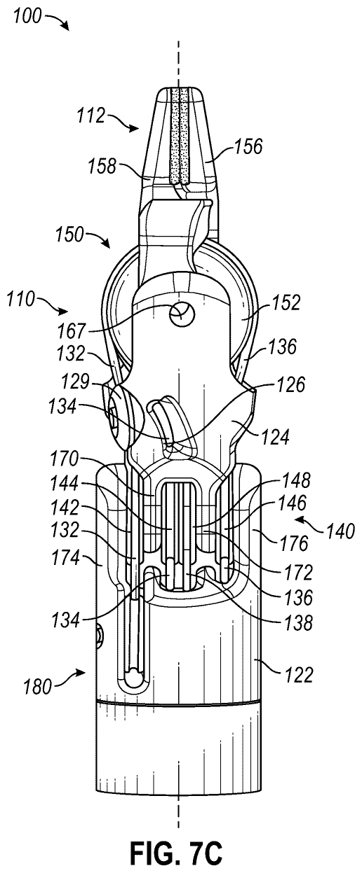

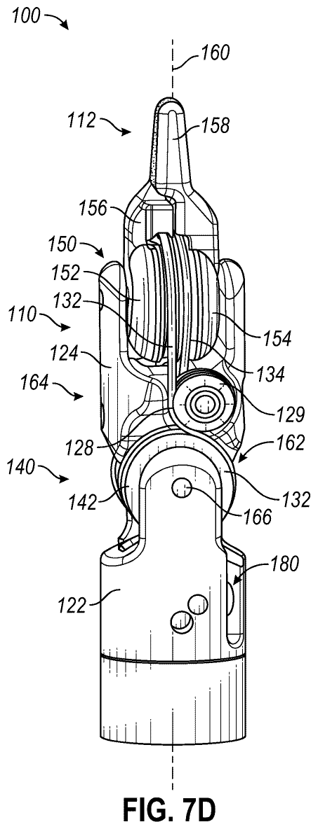

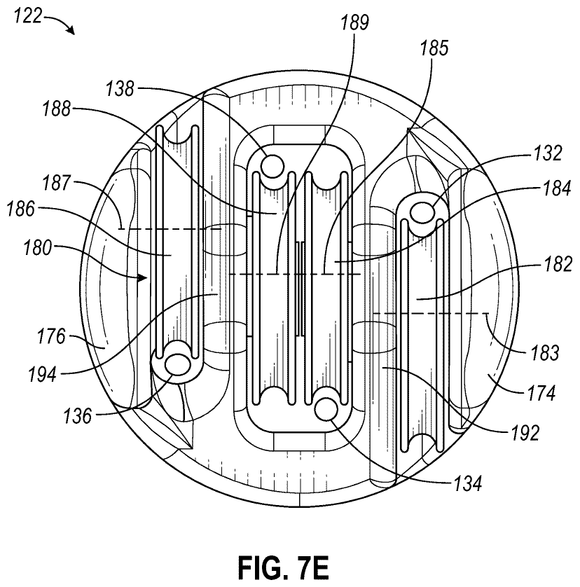

FIGS. 7A-7E illustrate an example of a medical instrument (100) that may be incorporated into a robotic medical system, such as either of the robotic systems (10, 28) described above. For example, instrument (100) may be readily incorporated into either robotic system (10) in place of any of instruments (14).

FIG. 7A is a perspective view of the medical instrument (100). FIG. 7B is another perspective view of the medical instrument (100), shown with a distal clevis (124) illustrated as transparent so as to visualize certain internal features thereof. FIG. 7C is a first side view of the medical instrument (100). FIG. 7D is a second side view of the medical instrument (100). FIG. 7E is a top view of a proximal clevis (122) of the medical instrument (100).

As shown in FIG. 7A, in the illustrated example, the medical instrument (100) includes an elongated shaft (102) extending to a distal end (104). A wrist (110) is positioned at the distal end (104) of the elongated shaft (102). The wrist (110) is also connected to an end effector (112), which is a grasper in the illustrated example.

In the illustrated example, the wrist (110) comprises a proximal clevis (122) and a distal clevis (124). The proximal clevis (122) can be attached to the distal end (104) of the elongated shaft (102). In the illustrated example, the distal clevis (124) is pivotally attached to the proximal clevis (122) by an axle (166) which extends through the distal clevis (124) and the proximal clevis (122). The distal clevis (124) can rotate about an axis of the axle (166) relative to the proximal clevis (122).

As best seen in FIG. 7C, the proximal clevis (122) can include a first proximal clevis support leg (174) and a second proximal clevis support leg (176). The axle (166) can extend through the first proximal clevis support leg (174) and the second proximal clevis support leg (176) of the proximal clevis (122). Similarly, the distal clevis (124) can include a first distal clevis support leg (170) and a second distal clevis support leg (172). The axle (166) extends through the first distal clevis support leg (170) and the second distal clevis support leg (172) of the distal clevis (124).

As shown in FIGS. 7A-7D, the medical instrument (100) includes a plurality of proximal pulleys (140) and a plurality of distal pulleys (150) positioned in the wrist (110). As best seen in FIGS. 7A-7C, the proximal pulleys (140) can be positioned on the axle (166) that connects the proximal clevis (122) and the distal clevis (124). In the illustrated example, the proximal pulleys (140) include a first outer proximal pulley (142), a first inner proximal pulley (144), a second outer proximal pulley (146), and a second inner proximal pulley (148). The first outer proximal pulley (142), the first inner proximal pulley (144), the second outer proximal pulley (146), and the second inner proximal pulley (148) can each be positioned on the axle (166) such that they can rotate about the axle (166).

As seen in FIGS. 7A-7D, the distal pulleys (150) can be positioned on an axle (167). The axle (167) can extend through the distal clevis (124) as shown. In the illustrated example, the distal pulleys (150) include a first distal pulley (152) and a second distal pulley (154) mounted on the axle (167).

The pitch axle (166) and the yaw axle (167) can be oriented at an angle with respect to each other. In the illustrated example, the pitch axle (166) and the yaw axle (167) are orthogonal. Accordingly, the pitch plane and the yaw plane can also be orthogonal to each other.

The end effector (112) of the medical instrument (100) can be formed by a first jaw member (156) and a second jaw member (158), which may also be referred to as a first jaw (156) and a second jaw (158), respectively. The first jaw member (156) can be connected to the first distal pulley (152) and the second jaw member (158) can be connected to the second distal pulley (154). The orientation of the end effector (112) can be controlled by rotating the first distal pulley (152) and the second distal pulley (154) in the same direction about the axle (167). For example, by rotating both of the first distal pulley (152) and the second distal pulley (154) in the same direction about the axle (167), the yaw of the end effector (112) can be adjusted. The end effector (112) can be actuated (e.g., opened or closed in the case of the illustrated grasper) by rotating the first distal pulley (152) and the second distal pulley (154) in the opposite directions about the axle (167).

The medical instrument (100) can include a plurality of pull wires (130) that can be actuated (e.g., pulled or tensioned) to control the three degrees of freedom of the medical instrument (100) (pitch, yaw, and actuation). As shown in FIGS. 7A-7D, the plurality of pull wires (130) are engaged with the proximal pulleys (140) and the distal pulleys (150). In the illustrated example, the plurality of pull wires (130) include a first pull wire segment (132), a second pull wire segment (134), a third pull wire segment (136), and a fourth pull wire segment (138) which are routed along various paths through the wrist (110).

For example, in the illustrated example, the first pull wire segment (132) engages the first outer proximal pulley (142) and the first distal pulley (152). Actuation of the first pull wire segment (132) can be associated with closing the first jaw member (156). The second pull wire segment (134) can be engaged with the first inner proximal pulley (144) and the second distal pulley (154). The second pull wire segment (134) can be associated with opening the second jaw member (158). The third pull wire segment (136) can be engaged with the second outer proximal pulley (146) and second distal pulley (154). The third pull wire segment (136) can be associated with closing the second jaw member (158). The fourth pull wire segment (138) can be engaged with the second inner proximal pulley (148) and the first distal pulley (152). The fourth pull wire segment (138) can be associated with opening the first jaw member (156).

As shown in the figures, each of the first pull wire segment (132) and the fourth pull wire segment (138) can engage the first distal pulley (152), but on opposite sides. Similarly, each of the second pull wire segment (134) and the third pull wire segment (136) can engage the second distal pulley (154), but on opposite sides. In the illustrated example, each of the proximal pulleys (140) is only engaged by one of the pull wire segments. The first pull wire segment (132) engages the first outer proximal pulley (142) on the same side of the wrist (110) that the fourth pull wire segment (138) engages the second inner proximal pulley (148). Similarly, the second pull wire segment (134) engages the first inner proximal pulley (144) on the same side of the wrist (110) that the third pull wire segment (136) engages the second outer, proximal pulley (146). At the proximal pulleys (140), the first and fourth pull wire segments (132, 138) are positioned on an opposite side of the wrist (110) than the second and third pull wire segments (134, 136).

As best seen in FIG. 7B, which illustrates the distal clevis (124) as transparent, the plurality of pull wires (130) are redirected between proximal pulleys (140) and distal pulleys (150). To accomplish the redirection, the wrist (110) of the instrument (100) includes hybrid redirect surfaces. Specifically, in the illustrated example, the wrist (110) includes a pair of static redirect surfaces and a pair of dynamic redirect surfaces positioned between proximal pulleys (140) and distal pulleys (150). As shown in FIG. 7B, the pair of static redirect surfaces include a first static redirect surface (126) and a second static redirect surface (133). The first static redirect surface (126) and the second static redirect surface (133) can each be an angled or curved surface formed in or on the distal clevis (124). An example is visible in FIG. 7C, which shows the static redirect surface (126). The pair of dynamic redirect surfaces include a first dynamic redirect surface (128) and a second dynamic redirect surface (131). Each of the first dynamic redirect surface (128) and the second dynamic redirect surface (131) can comprise a surface of a redirect pulley, such as the first redirect pulley (129) and the second redirect pulley (135) that are illustrated in the figures.

The plurality of pull wires (130) are redirected by the static redirect surfaces (126, 133) and the dynamic redirect surfaces (128, 131). In the illustrated example, the first pull wire segment (132) engages the first dynamic redirect surface (128). The second pull wire segment (134) engages the first static redirect surface (126). The third pull wire segment (136) engages the second dynamic redirect surface (131). The fourth pull wire segment (138) engages the second static redirect surface (133).

Thus, in this example, the first and third pull wire segments (132, 136), which are associated with closing the end effector (112) are redirected using the dynamic redirect surfaces (128, 131) of the redirect pulleys (129, 135), respectively. The second and fourth pull wire segments (134, 138), which are associated with opening the end effector (112) are redirected using the static redirect surfaces (126, 133), respectively.

The medical instrument (100) also includes shaft redirect pulleys (180) positioned in the proximal clevis (122) and/or within the elongated shaft (102). The shaft redirect pulleys (180) are best seen in FIG. 7E which is a top down view of the proximal clevis (122). As shown, the shaft redirect pulleys (180) include a first outer shaft redirect pulley (182), a first inner shaft redirect pulley (184), a second outer shaft redirect pulley (186), and second inner shaft redirect pulley (188). In the illustrated example, the shaft redirect pulleys (180) are in a staggered position. That is, as shown in FIG. 7E, the first outer shaft redirect pulley (182) is positioned on first axis (183) and the first inner shaft redirect pulley (184) is positioned on second axis (185). The first and second axes (183, 185) are not coaxial (in the illustrated example). The second inner shaft redirect pulley (188) is positioned on a third axis (189). In the illustrated example the third axis (189) is coaxial with second axis (185). The second outer shaft redirect pulley (186) is positioned on fourth axis (187). In the illustrated example, the fourth axis (187) is not coaxial with the first, second, or third axes (183, 185, 189). The proximal clevis (122) also comprises a first proximal clevis support wall (192) and a second proximal clevis support wall (194). The first proximal clevis support wall (192) is positioned between the first inner and outer shaft redirect pulleys (182, 184). The second proximal clevis support wall (194) is positioned between the second inner and outer shaft redirect pulleys (186, 184). The first proximal clevis support leg (174) and the second proximal clevis support leg (176) are also shown in FIG. 7E.

By way of further example, medical instrument (100) may be configured and operable in accordance with at least some of the teachings of U.S. Pub. No. 2020/0405423, entitled “Medical Instruments Including Wrists with Hybrid Redirect Surfaces,” published Dec. 31, 2020, the disclosure of which is incorporated by reference herein, in its entirety.

### III. Needle Driver Instrument With Grooved End Effector

In some instances, it may be desirable to configure medical instrument (200) as a needle driver instrument that is capable of providing effective gripping of both a needle and a suture. For example, it may be desirable for medical instrument (200) to include an end effector (212) distally positioned on medical instrument (200) that is capable of providing effective gripping of the needle when in a needle-gripping mode, as well as effective gripping of the suture when in a suture-gripping mode, while reducing risk of damaging the suture. The end effector (212) of the medical instrument (200) can be formed by a first jaw member (256) and a second jaw member (258). The first jaw member (256) can be connected to the first distal pulley (152) and the second jaw member (258) can be connected to the second distal pulley (154). The orientation of the end effector (212) can be controlled by rotating the first distal pulley (152) and the second distal pulley (154) in the same direction about the axle (167). For example, by rotating both of the first distal pulley (152) and the second distal pulley (154) in the same direction about the axle (167), the yaw of the end effector (212) can be adjusted. The end effector (212) can be actuated (e.g., opened or closed in the case of the illustrated grasper) by rotating the first distal pulley (152) and the second distal pulley (154) in the opposite directions about the axle (167).

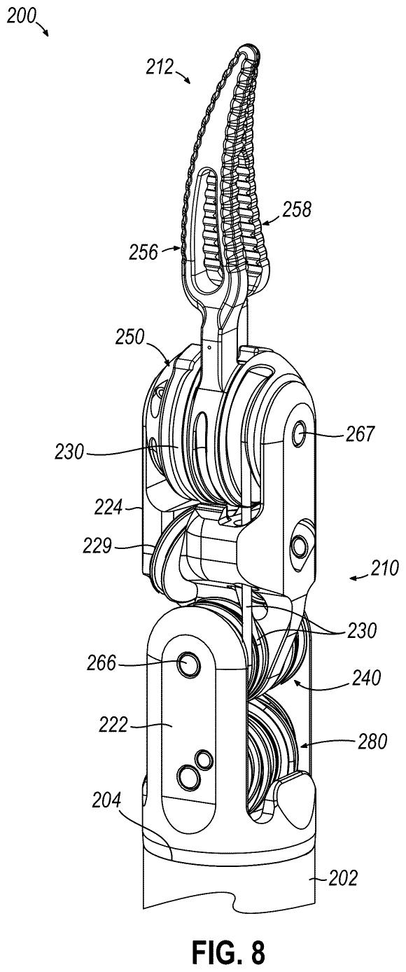

The medical instrument (200) can include a plurality of pull wires (230) that can be actuated (e.g., pulled or tensioned) to control the three degrees of freedom of the medical instrument (200) (pitch, yaw, and actuation). As shown in FIG. 8, the plurality of pull wires (230) are engaged with the proximal pulleys (240) and the distal pulleys (250). In the illustrated example, the plurality of pull wires (230) include a first pull wire segment (232), a second pull wire segment (234), a third pull wire segment (236), and a fourth pull wire segment (238) which are routed along various paths through the wrist (210).

For example, in the illustrated example, the first pull wire segment (232) engages the first outer proximal pulley (242) and the first distal pulley (252). Actuation of the first pull wire segment (232) can be associated with closing the first jaw member (256). The second pull wire segment (234) can be engaged with the first inner proximal pulley (244) and the second distal pulley (254). The second pull wire segment (234) can be associated with opening the second jaw member (258). The third pull wire segment (236) can be engaged with the second outer proximal pulley (246) and second distal pulley (254). The third pull wire segment (236) can be associated with closing the second jaw member (258). The fourth pull wire segment (238) can be engaged with the second inner proximal pulley (248) and the first distal pulley (252). The fourth pull wire segment (238) can be associated with opening the first jaw member (256).

As shown in the figures, each of the first pull wire segment (232) and the fourth pull wire segment (238) can engage the first distal pulley (252), but on opposite sides. Similarly, each of the second pull wire segment (234) and the third pull wire segment (236) can engage the second distal pulley (254), but on opposite sides. In the illustrated example, each of the proximal pulleys (240) is only engaged by one of the pull wire segments. The first pull wire segment (232) engages the first outer proximal pulley (242) on the same side of the wrist (210) that the fourth pull wire segment (238) engages the second inner proximal pulley (248). Similarly, the second pull wire segment (234) engages the first inner proximal pulley (244) on the same side of the wrist (210) that the third pull wire segment (236) engages the second outer, proximal pulley (246). At the proximal pulleys (240), the first and fourth pull wire segments (232, 238) are positioned on an opposite side of the wrist (210) than the second and third pull wire segments (234, 236).

FIGS. 8-21 show a portion of an example of medical instrument (200). Medical instrument (200) may be similar to medical instrument (100) described above, except as otherwise described below. In this regard, medical instrument (200) may be incorporated into a robotic medical system, such as either of the robotic systems (10, 28) described above. For example, instrument (200) may be readily incorporated into either robotic system (10) in place of any of instruments (14). Instrument (200) of the present example includes an elongated shaft (202) extending along a longitudinal axis to a distal end (204), a wrist (210) positioned at distal end (204), and an end effector in the form of a dual mode needle driver (212) connected to wrist (210).

As shown in FIG. 8, wrist (210) comprises a proximal clevis (222) and a distal clevis (224) similar to proximal clevis (122) and distal clevis (224) described above, respectively. In this regard, proximal clevis (222) is attached to distal end (204) of elongated shaft (202), and distal clevis (224) is pivotally attached to proximal clevis (222) by a pitch axle (266) such that distal clevis (224) can rotate about an axis of axle (266) relative to proximal clevis (222). Medical instrument (200) also includes a plurality of proximal pulleys (240) and a plurality of distal pulleys (250) similar to proximal pulleys (140) and distal pulleys (150) described above, respectively. In this regard, proximal pulleys (240) are positioned on axle (266), and distal pulleys (250) are positioned on a yaw axle (267).

In the example shown, dual mode needle driver (212) of medical instrument (200) includes a first jaw member (256) and a second jaw member (258) connected to respective distal pulleys (250) in a manner similar to that described above in connection with first and second jaw members (156, 158), such that dual mode needle driver (212) can be actuated (e.g., opened or closed) by rotating the respective distal pulleys (250) in opposite directions about axle (267).

Medical instrument (200) also includes a plurality of pull wires (230) similar to pull wires (130) described above. In this regard, pull wires (230) can be actuated (e.g., pulled or tensioned) to control the three degrees of freedom of medical instrument (200) (pitch, yaw, and actuation). As shown in FIG. 8, the plurality of pull wires (230) are engaged with proximal pulleys (240) and distal pulleys (250), with various segments of pull wires (230) routed along various paths through wrist (210) in a manner similar to that described above in connection with pull wires (230). In the example shown, the plurality of pull wires (230) are redirected between proximal pulleys (240) and distal pulleys (250) via one or more dynamic redirect surfaces defined by one or more redirect pulleys (229) (one shown), which may be similar to redirect pulleys (129, 135) described above, and/or via one or more static redirect surfaces (not shown), which may be similar to static redirect surfaces (126, 133) described above. Medical instrument (200) also includes shaft redirect pulleys (280) positioned in proximal clevis (222) and/or within elongated shaft (202), similar to shaft redirect pulleys (180) described above.

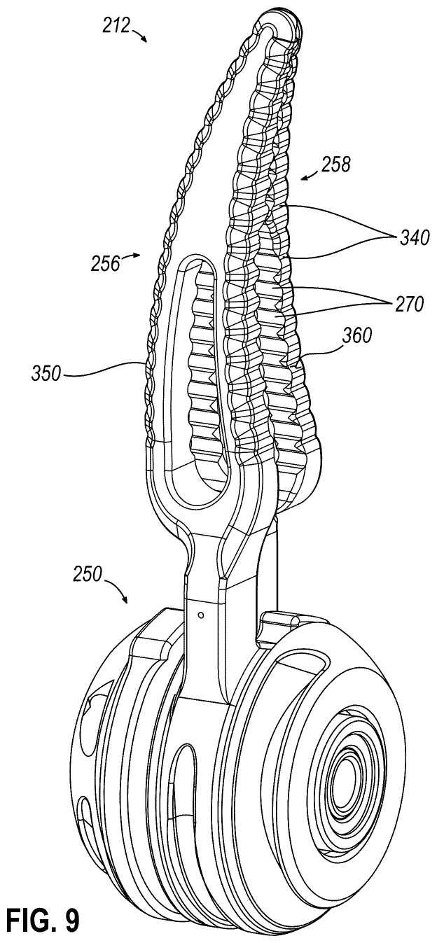

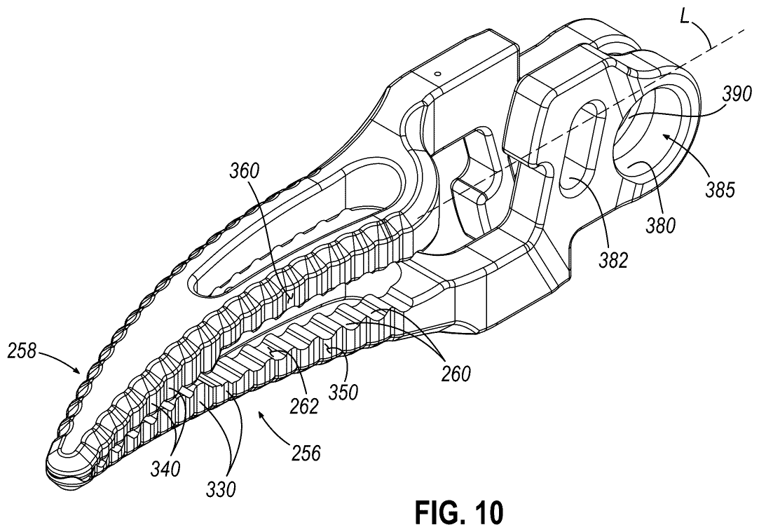

Referring primarily to FIGS. 9-12, each jaw member (256, 258) of end effector (212) is more particularly in the present example formed as a clamp arm (256, 258) extending distally relative to the respective distal pulley (250) and having a distal, laterally-inwardly facing surface, labelled as internal surfaces (262 (shown in FIGS. 10), 272 (shown in FIG. 9)). Jaw members (256, 258) are arranged such that internal surfaces (262, 272) are configured to be opposed from each other when jaw members (256, 258) are pivoted toward each other to define at least one closed state of end effector (212).

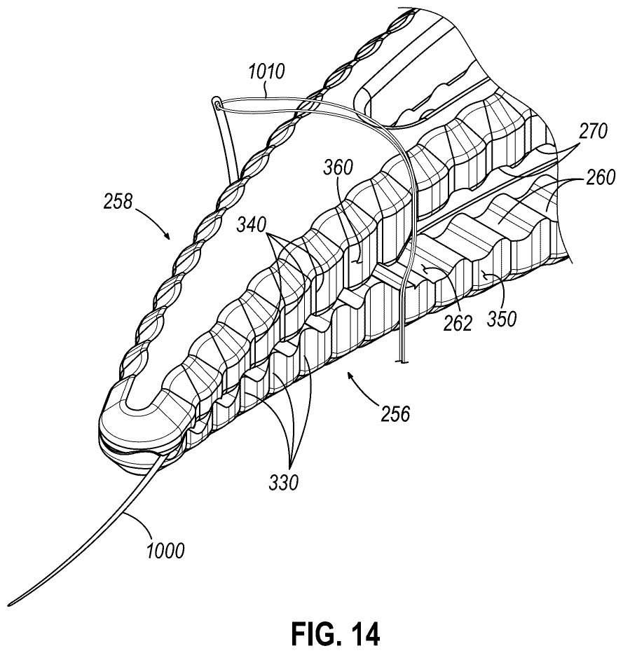

Internal surfaces (262, 272) each longitudinally include one or more grooves (260, 270). Grooves (260) include a crest (261) and a valley (263), whereas grooves (270) include a crest (271) and a valley (273). Each valley (263, 273) of grooves (260, 270) is sized to receive, and configured to accept, a needle (1000) (shown in FIG. 14) or a suture thread (1010) (shown in FIG. 14), therein. Each valley (263, 273) is flanked longitudinally by a pair of crests (261, 263), and each crest (261, 263) of each groove (260, 270) physically inhibits needle (1000) and/or suture thread (1010) from traversing along the internal surfaces (262, 272) in a longitudinal direction while needle (1000) and/or suture thread (1010) is in a valley (263, 273) of a grooves (260, 270) during operation of medical instrument (200).

Each jaw member (256, 258) further includes side surfaces (350, 360), generally perpendicular to internal surfaces (262, 272), respectively. Each side surface (350, 360) longitudinally defines one or more grooves (330, 340). Grooves (330) include a crest (331) and a valley (333), whereas grooves (340) include a crest (341) and a valley (343). Each valley (333, 343) of grooves (330, 340) is sized to receive, and configured to accept, a needle (1000) (shown in FIG. 14) or a suture thread (1010) (shown in FIG. 14), therein. Each valley (333, 343) is flanked longitudinally by a pair of crests (331, 341), and each crest (331, 341) of grooves (330, 340) physically inhibits needle (1000) and/or suture thread (1010) from traversing along the side surfaces (350, 360) in a longitudinal direction while needle (1000) and/or suture thread (1010) is in a valley (333, 343) of a groove (330, 340) during operation of medical instrument (200).

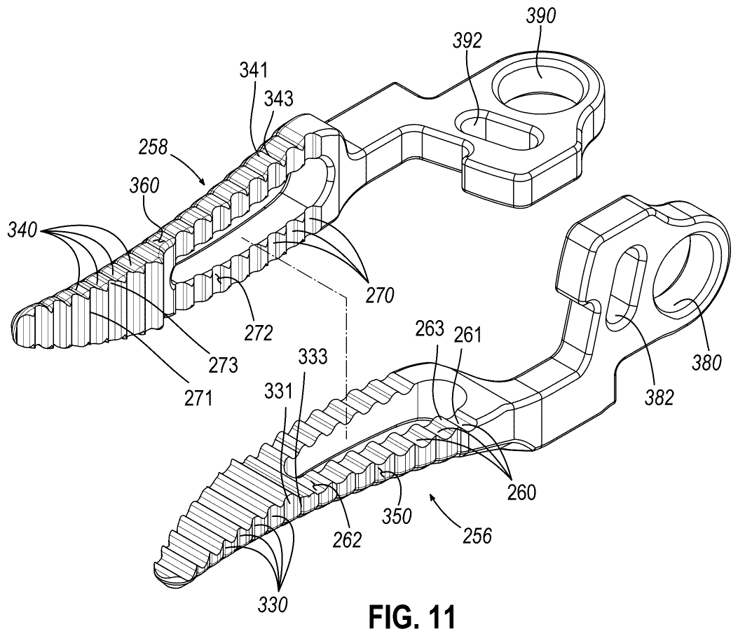

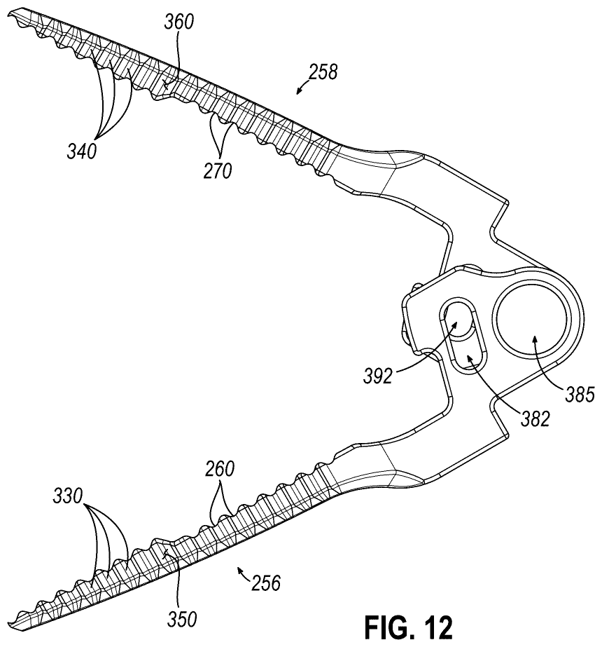

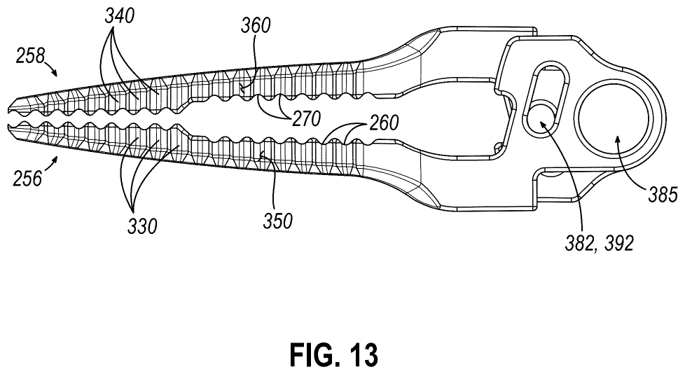

With reference to the proximal end of each of the jaws (256, 258), apertures (380, 390), respectively, are located thereon. As shown in FIG. 11, each of the jaws (256, 258) may be brought together such that apertures (380, 390) align and form a channel (385). Jaws (256, 258) may be coupled relative to each other as shown and discussed with respect to medical instrument (100) (see FIG. 7A) and, in one example, jaws (256, 258) may be coupled to into axle (250), for example by inserting a bolt through channel (385), as shown assembled in FIG. 9, to form the end effector (212). Furthermore, through holes (382, 392) (shown in FIGS. 12 and 13) are aligned such that a bolt may be inserted simultaneously through each of through holes (382, 392). As will be described in more detail below, jaws (256, 258) are pivotable relative to each other between an open state and a closed state. While in the closed state, the first interior surface and the second interior surface are configured to cooperate with each other to securely grip needle (1000) and/or suture thread (1010).

Jaws (256, 258) are pivotable relative to each other via actuation around a pivot point within channel (385). In some examples, a bolt received within through holes (382, 392) simultaneously prevents further opening or closing of jaws (256, 258) relative to each other. For example, FIG. 12 shows jaws (256, 258) in the open position, wherein the distal portion of each of jaws (256, 258) are spaced further apart from each other relative to the closed position, as shown in FIG. 13. Through holes (382, 392) are positioned such that, in the open state shown in FIG. 12, when a bolt extends through each of the through holes (382, 392) simultaneously, the bolt is located at the upper end of through hole (382) and the lower end of through hole (392). In the open state, the bolt contacts the upper end of through hole (382) and the lower end of through hole (392), preventing further opening of jaws (256, 258). In contrast, in the closed state, the bolt is located at the lower end of through hole (382) and the upper end of through hole (392). The bolt contacts the lower end of through hole (382) and the upper end of through hole (392), preventing further closing of jaws (256, 258). In an example, as shown in FIG. 13, internal surfaces (262, 272) are spaced apart from each other in the closed state, and do not contact each other. In other examples, internal surfaces (262, 272) may contact each other in the closed state (not shown).

FIG. 14 shows a needle (1000) and suture thread (1010) being handled by the end effector (212). Suture thread (1010) is wrapped around the jaws (256, 258), wherein a portion of suture thread (1010) is accepted within grooves (330, 340) defined by the side surfaces (350, 360). Specifically, the suture thread (1010) is accepted in a valley (333, 343) of the grooves (330, 340), and crests (331, 341) of the grooves (330, 340) physically reduce movement of the suture thread (1010) in a longitudinal direction of a longitudinal axis (L) of the end effector (212). While FIG. 14 shows suture thread (1010) in only one groove (330, 340) along each jaw's (256, 258) side surfaces (350, 360), it is possible for the suture thread (1010) to be wrapped around one or more jaws (256, 258) such that the suture thread (1010) is accepted within a plurality of grooves (330, 340) in any combination or sub-combination. As furthermore shown in FIG. 14, needle (1000) is grasped by jaws (256, 258) in the closed state, and is readily manipulated by end effector (212).

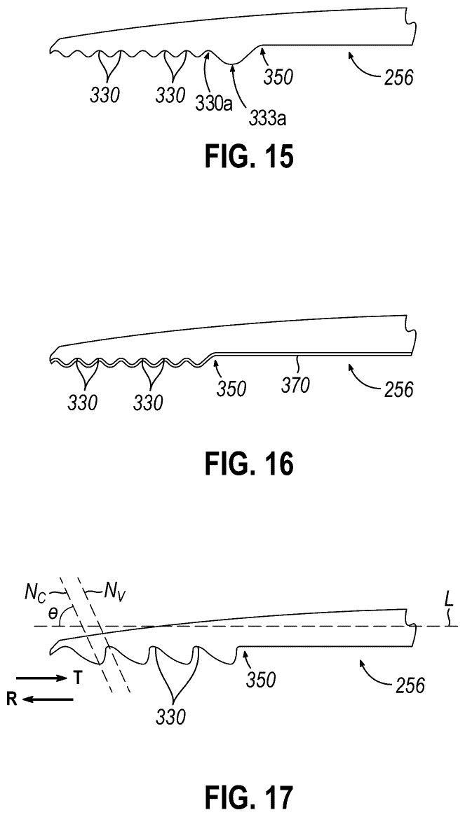

While FIGS. 9-14 show jaws (256, 258) having uniformly spaced grooves (259, 296), FIGS. 15-20 show alternative examples of groove (259) orientations. While FIGS. 15-20 show jaw (256) specifically, the same configurations shown in FIGS. 15-20 are applicable to jaw (258). Each of jaws (256, 258) have groove (259, 296) configurations independent of each other.

FIG. 15 shows an example of jaw (256) wherein the grooves (330) are distally located along side surface (350). The most proximal groove (330a) includes a most proximal crest (331a). The most proximal crest (331a) has a larger amplitude than all other crests (331) along side surface (350). While not limited to this configuration, and not the only advantage of this configuration, an advantage of having differently sized crests (331) along side surface (350) is that an operator of medical instrument (200) has a meaningful choice of which groove (330) to receive suture thread (1010). For example, it may be preferrable for suture thread (1010) to be received in proximal groove (330a) if suture thread (1010) is relatively thick such that the increased amplitude of proximal crest (331a) better reduces longitudinal movement of suture thread (1010) relative to the other grooves (330) along side surface (350). It will be appreciated that such different sized grooves (330a) and crests (331) may be incorporated into any medical instrument, such as medical instrument (200), as discussed above.

FIG. 16 shows an example of jaw (256) wherein side surface (350) is coated with a surface treatment material (370). The surface treatment material (370) is configured to increase the coefficient of friction of side surface (350) relative to side surface (350) without the surface treatment material (370). In some examples, the surface treatment material (370) may be tungsten carbide, alumina-zirconia, aluminum oxide, and combinations thereof. Surface treatment material (370) may be coated onto a portion or the entire longitudinal length of side surface (350). The increase in the coefficient of friction of side surface (350) from the application of surface treatment material (370) may assist in further reducing longitudinal movement of suture thread (1010) by increasing friction between the suture thread (1010) and the side surface (350). It will be appreciated that such surface treatment materials (370) may be incorporated into any medical instrument, such as medical instrument (200), as discussed above.

FIG. 17 shows an example of jaw (256), wherein the grooves (330) are angled relative to side surface (350) and longitudinal axis (L). Particularly, a crest axis normal to a crest (Nc) and a valley axis normal to a valley (Nv) of a representative groove (330) intersects with longitudinal axis (L) at an angle (θ), wherein the angle (θ) is different than 90 degrees. In the example shown in FIG. 17, angle (θ) is less than 90 degrees, but alternatively, angle (θ) may be greater than 90 degrees. One example of a non-limiting advantage to this configuration is that, when suture thread (1010) is accepted within a groove (330), the angled nature of the grooves (330) may further assist in reducing longitudinal movement of suture thread (1010) when received into groove (330). It will be appreciated that such angled crest (Nc) and valley (Nv) may be incorporated into any medical instrument, such as medical instrument (200), as discussed above.

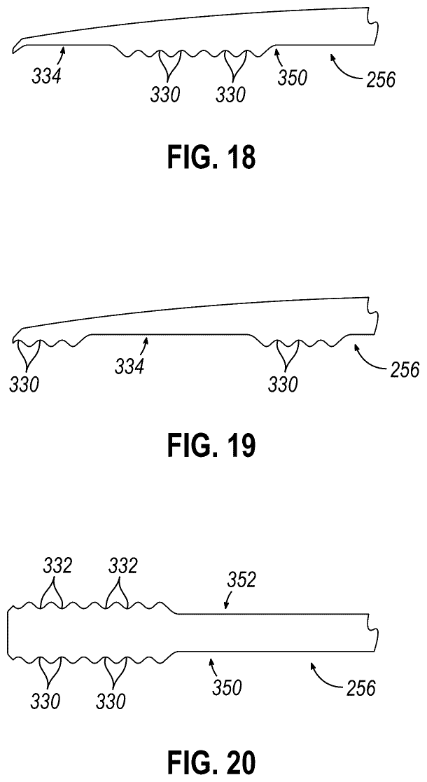

FIG. 18 shows an example of jaw (256) where grooves (330) do not extend to the distal tip of jaw (256). Rather, side surface (350) includes a non-grooved portion (334). Similarly, FIG. 19 shows a non-grooved portion (334) of side surface (350) between two portions of grooves (330). One example of a non-limiting advantage of these configurations may be that suture thread (1010) may be desired to be longitudinally moveable, but only on the non-grooved portion (334) of side surface (350). The non-grooved portion (342) of side surface (350) increases the customizable use of end effector (212) when manipulating needle (1000) and/or suture thread (1010). It will be appreciated that non-grooved portions (334) may be incorporated into any medical instrument, such as medical instrument (200), as discussed above.

FIG. 20 shows an example of jaw (256), which includes another one or more grooves (332) along a second side surface (352), the second side surface (352) opposite side surface (350). The inclusion of grooves (332) along second side surface (352) allows an operator of end effector (212) to use either side surface (350, 352) of jaw (256) to receive suture thread (1010) within one or more grooves (330, 332). This configuration may be duplicated or solely present on jaw (258) as well. Like grooves (330), grooves (332) physically interfere with longitudinal movement of suture thread (1010) along side surface (352), which allows an operator of end effector (212) additional freedom of choice in how to manipulate needle (1000) and/or suture thread (1010). It will be appreciated that grooves (332) along second side surface (352) may be incorporated into any medical instrument, such as medical instrument (200), as discussed above.

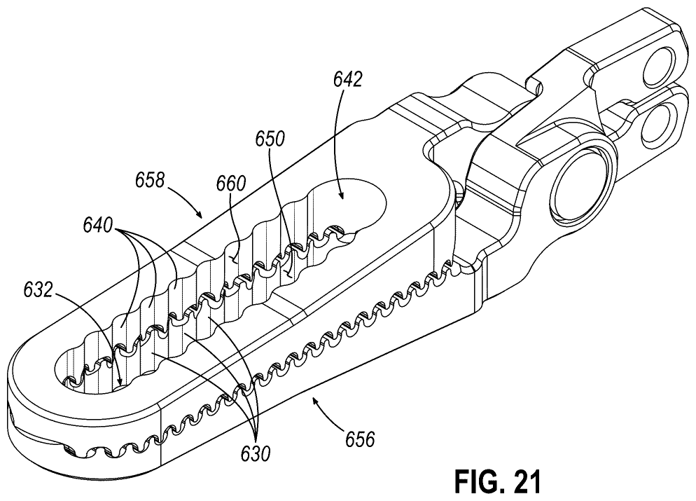

FIG. 21 shows another example of an end effector. Jaws (656, 658) are pivotable relative to each other between a closed state and an open state, similar to the jaws (256, 258) described in previous examples. Jaws (656, 658) include through holes (632, 642) respectively, each extending in a direction of the other jaw (658, 656) in the closed state. Each through hole (632, 642) is defined by jaws (656, 658) respectively via hole surfaces (650, 660). Hole surfaces (650, 660) define grooves (630, 640), respectively, running circumferentially along hole surfaces (650, 660). Grooves (630, 640) include crests and valleys, wherein the valleys are configured to accept a suture thread therein. In such a configuration, suture thread (1010) may be received in through hole (632) and/or through hole (642) and accepted into a groove (630, 640). Once accepted into a valley of a groove (630, 640), crests of grooves (630, 640) adjacent the valley physically interfere with longitudinal movement of suture thread (1010), similar to grooves (330, 340) as described above. It will be appreciated that these features associated with jaws (656, 658) may be incorporated into any medical instrument, such as medical instrument (200), as discussed above.

### IV. Illustrative Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: (a) a first jaw, including: (i) a first interior surface, and (ii) a first side surface, the first side surface defining a first groove configured to accept a suture thread within, and inhibit the suture thread from traversing along the first side surface in a longitudinal direction; and (b) a second jaw, including: (i) a second interior surface opposite the first interior surface, and (ii) a second side surface, the second side surface defining a second groove configured to accept the suture thread within, and inhibit the suture thread from traversing along the second side surface in the longitudinal direction, the first and second jaws being pivotable relative to each other between: (i) an open state, and (ii) a closed state in which the first interior surface and the second interior surface are configured to cooperate with each other to securely grip a needle or the suture thread.

### Example 2

The surgical instrument of Example 1, wherein the first interior surface and the second interior surface are configured to be spaced apart from each other when the first jaw and the second jaw are in the closed state.

### Example 3

The surgical instrument of any one or more of Examples 1 through 2, wherein the first groove is one of a first plurality of grooves defined longitudinally along the first side surface.

### Example 4

The surgical instrument of Example 3, wherein the second groove is one of a second plurality of grooves defined longitudinally along the second side surface.

### Example 5

The surgical instrument of any one or more of Examples 1 through 4, wherein the first side surface is coated with a surface treatment material configured to increase a coefficient of friction of the first side surface relative to the first side surface without the surface treatment material coated thereon.

### Example 6

The surgical instrument of Example 5, wherein the second side surface is coated with the surface treatment material configured to increase a coefficient of friction of the second side surface relative to the second side surface without the surface treatment material coated thereon.

### Example 7

The surgical instrument of Example 3, wherein at least one pair of the first plurality of grooves is separated by a non-grooved portion longitudinally along the first side surface.

### Example 8

The surgical instrument of Example 3, wherein at least one of the first plurality of grooves comprises a crest and a valley, wherein the crest comprises a crest axis normal to the crest and the valley comprises a valley axis normal to the valley, the crest axis being parallel to the valley axis.

### Example 9

The surgical instrument of Example 8, wherein the crest axis and the valley axis each form a less than 90 degree angle with a longitudinal axis through the first jaw.

### Example 10

The surgical instrument of Example 8, wherein the crest axis and the valley axis each form about a 90 degree angle with a longitudinal axis through the first jaw.

### Example 11

The surgical instrument of Example 3, wherein the first plurality of grooves each comprise a crest and a valley, wherein a proximal crest has a greater amplitude than each of the first plurality of grooves.

### Example 12

The surgical instrument of any one or more of Examples 1 through 11, wherein the first jaw further comprises a first through hole defined by the first jaw.

### Example 13

The surgical instrument of any one or more of Examples 1 through 12, wherein the first jaw comprises a third side surface, the third side surface opposite the first side surface, the third side surface defining a third groove.

### Example 14

The surgical instrument of Example 13, wherein the third groove is one of a plurality of grooves defined by the third side surface.

### Example 15

The surgical instrument of Example 13, wherein the second jaw comprises a fourth side surface, the fourth side surface opposite the second side surface, the fourth side surface defining a fourth groove.

### Example 16

The surgical instrument of Example 15, wherein the fourth groove is one of a plurality of grooves defined by the fourth side surface.

### Example 17

A surgical instrument, comprising: (a) a first jaw, including: (i) a first interior surface, and (ii) a first through hole defined by the first jaw, the first through hole comprising a first through hole surface defining a first groove configured to accept a suture thread therein, and inhibit the suture thread from traversing along the first through hole surface in a longitudinal direction; and (b) a second jaw, including: (i) a second interior surface opposite the first interior surface, and (ii) a second through hole defined by the second jaw, the second through hole comprising a second through hole surface defining a second groove configured to accept the suture thread therein, and inhibit the suture thread from traversing along the first through hole surface in a longitudinal direction; and the first and second jaws being pivotable relative to each other between: (i) an open state, and (ii) a closed state in which the first interior surface and the second interior surface are configured to cooperate with each other to securely grip a needle or the suture thread.

### Example 18

The surgical instrument of Example 17, wherein the first through hole surface is coated with a surface treatment material configured to increase a coefficient of friction of the first through hole surface relative to the first through hole surface without the surface treatment material coated thereon.

### Example 19

The surgical instrument of any one or more of Examples 17 through 18, wherein the first groove is one of a first plurality of grooves, the first plurality of grooves being continuous along the first through hole surface.

### Example 20

The surgical instrument of any one or more of Examples 17 through 19, wherein a side surface of at least one of the first or second jaws is coated with a surface treatment material configured to increase a coefficient of friction of the side surface relative to the first side surface without the surface treatment material coated thereon.

### Example 21

A surgical instrument, comprising: (a) a shaft extending along a longitudinal axis to a distal end; and (b) an end effector operatively coupled to the distal end of the shaft, the end effector including a pair of jaws, the pair of jaws being pivotable relative to each other for gripping at least one surgical object, at least one jaw of the pair of jaws including: (i) a side surface, the side surface defining a groove configured to accept a suture thread therein, and inhibit the suture thread from traversing along the side surface in a longitudinal direction.

### V. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof are placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument, comprising:
(a) a first jaw, including:
(i) a first interior surface, and
(ii) a first side surface, the first side surface defining a first groove configured to accept a suture thread within, and inhibit the suture thread from traversing along the first side surface in a longitudinal direction; and
(b) a second jaw, including:
(i) a second interior surface opposite the first interior surface, and
(ii) a second side surface, the second side surface defining a second groove configured to accept the suture thread within, and inhibit the suture thread from traversing along the second side surface in the longitudinal direction,
the first and second jaws being pivotable relative to each other between:
(i) an open state, and
(ii) a closed state in which the first interior surface and the second interior surface are configured to cooperate with each other to securely grip a needle or the suture thread.

2. The surgical instrument of claim 1, wherein the first interior surface and the second interior surface are configured to be spaced apart from each other when the first jaw and the second jaw are in the closed state.

3. The surgical instrument of claim 1, wherein the first groove is one of a first plurality of grooves defined longitudinally along the first side surface.

4. The surgical instrument of claim 3, wherein the second groove is one of a second plurality of grooves defined longitudinally along the second side surface.

5. The surgical instrument of claim 1, wherein the first side surface is coated with a surface treatment material configured to increase a coefficient of friction of the first side surface relative to the first side surface without the surface treatment material coated thereon.

6. The surgical instrument of claim 5, wherein the second side surface is coated with the surface treatment material configured to increase a coefficient of friction of the second side surface relative to the second side surface without the surface treatment material coated thereon.

7. The surgical instrument of claim 3, wherein at least one pair of the first plurality of grooves is separated by a non-grooved portion longitudinally along the first side surface.

8. The surgical instrument of claim 3, wherein at least one of the first plurality of grooves comprises a crest and a valley, wherein the crest comprises a crest axis normal to the crest and the valley comprises a valley axis normal to the valley, the crest axis being parallel to the valley axis.

9. The surgical instrument of claim 8, wherein the crest axis and the valley axis each form a less than 90 degree angle with a longitudinal axis through the first jaw.

10. The surgical instrument of claim 8, wherein the crest axis and the valley axis each form about a 90 degree angle with a longitudinal axis through the first jaw.

11. The surgical instrument of claim 3, wherein the first plurality of grooves each comprise a crest and a valley, wherein a proximal crest has a greater amplitude than each of the first plurality of grooves.

12. The surgical instrument of claim 1, wherein the first jaw further comprises a first through hole defined by the first jaw.

13. The surgical instrument of claim 1, wherein the first jaw comprises a third side surface, the third side surface opposite the first side surface, the third side surface defining a third groove.

14. The surgical instrument of claim 13, wherein the third groove is one of a plurality of grooves defined by the third side surface.

15. The surgical instrument of claim 13, wherein the second jaw comprises a fourth side surface, the fourth side surface opposite the second side surface, the fourth side surface defining a fourth groove.

16. A surgical instrument, comprising:
(a) a first jaw, including:
(i) a first interior surface, and
(ii) a first through hole defined by the first jaw, the first through hole comprising a first through hole surface defining a first groove configured to accept a suture thread therein, and inhibit the suture thread from traversing along the first through hole surface in a longitudinal direction; and
(b) a second jaw, including:
(i) a second interior surface opposite the first interior surface, and
(ii) a second through hole defined by the second jaw, the second through hole comprising a second through hole surface defining a second groove configured to accept the suture thread therein, and inhibit the suture thread from traversing along the first through hole surface in a longitudinal direction; and
the first and second jaws being pivotable relative to each other between:
(i) an open state, and
(ii) a closed state in which the first interior surface and the second interior surface are configured to cooperate with each other to securely grip a needle or the suture thread.

17. The surgical instrument of claim 16, wherein the first through hole surface is coated with a surface treatment material configured to increase a coefficient of friction of the first through hole surface relative to the first through hole surface without the surface treatment material coated thereon.

18. The surgical instrument of claim 16, wherein the first groove is one of a first plurality of grooves, the first plurality of grooves being continuous along the first through hole surface.

19. The surgical instrument of claim 16, wherein a side surface of at least one of the first or second jaws is coated with a surface treatment material configured to increase a coefficient of friction of the side surface relative to the first side surface without the surface treatment material coated thereon.

20. A surgical instrument, comprising:
(a) a shaft extending along a longitudinal axis to a distal end; and
(b) an end effector operatively coupled to the distal end of the shaft, the end effector including a pair of jaws, the pair of jaws being pivotable relative to each other for gripping at least one surgical object, at least one jaw of the pair of jaws including:
(i) a side surface, the side surface defining a groove configured to accept a suture thread therein, and inhibit the suture thread from traversing along the side surface in a longitudinal direction.
